(19) United States
(12) Patent Application Publication
(10) Pub. No.: US 2011/0123976 A1
(43) Pub. Date: May 26, 2011

(54) Biomarkers and identification methods for the early detection and recurrence prediction of breast cancer using NMR

(75) Inventors: M. Daniel Raftery, West Lafayette, IN (US); Vincent Asiago, Johnston, IA (US)

(73) Assignee: Purdue Research Foundation, West Lafayette, IN (US)

(21) Appl. No.: 12/925,054

(22) Filed: Oct. 13, 2010

Related U.S. Application Data
(60) Provisional application No. 61/250,917, filed on Oct. 13, 2009.
(60) Provisional application No. 61/285,672, filed on Dec. 11, 2009.

(51) Int. Cl. C12Q 1/00; C07C 53/06; C07D 233/64; C07C 229/36; C07D 233/88; C07C 229/08; C07H 3/02; C07C 229/22; C07C 279/14; C07C 229/26; C07C 323/58; C07C 229/24; C07D 207/16; C07C 59/01; G01N 33/68; G01R 33/44; G01N 33/487; G06F 17/18; G06F 19/00

(52) U.S. Cl. 435/4; 562/609; 548/339.1; 562/444; 548/321.5; 562/575; 536/1.11; 562/570; 562/560; 562/561; 562/563; 562/559; 562/568; 548/535; 562/588; 436/501; 702/19

(57) ABSTRACT

A method is provided for the parallel identification of one or more metabolite species within a biological sample. The method comprises analyzing the sample to produce a spectrum containing individual spectral peaks representative of the one or more metabolite species contained within the sample; subjecting each of the individual spectral peaks to a statistical pattern recognition analysis to identify the one or more metabolite species contained within the sample; and identifying the one or more metabolite species contained within the sample by analyzing the individual spectral peaks of the spectra.

### RELATED CASES

This application claims the benefit of U.S. Provisional Application Nos. 61/250,917, which was filed on Oct. 13, 2009, and 61/285,672, which was filed on Dec. 11, 2009, the disclosures of which are expressly incorporated herein by reference in their entirety.

### GOVERNMENT INTEREST

This disclosure was made in part with U.S. government support under grant reference number NIH/NIGMS 1R01GM085291-01 awarded by the National Institutes of Health. The Government has or may have certain rights in this disclosure.

### TECHNICAL FIELD

The present disclosure generally relates to small molecule biomarkers comprising metabolite species useful for the early detection of breast cancer, and for predicting the recurrence of breast cancer, and to methods for identifying and quantifying such biomarkers within biological samples.

### BACKGROUND

Current breast cancer detection methods often involve mammographic examinations, followed by biopsy procedures. However, mammographies often produce inaccurate results and thereby force many women to undergo unnecessary biopsies, which can be both painful and expensive. To combat the problems related to poor diagnostic accuracy for a number of diseases, research efforts have recently focused on metabolomics to diagnosis diseases through the identification of various biomarkers. Metabolomics provides a means to identify a subset of metabolites that differentiate sample populations, and to provide detailed information regarding biochemical status changes. The use of a single analytical method to uncover useful biomarkers for early disease detection, and in particular early breast cancer detection, has so far been unsuccessful.

### SUMMARY

The present disclosure is directed to, in one embodiment, a method for the parallel identification of one or more metabolite species within a biofluid. The method comprises producing a first spectrum by subjecting the biofluid to a nuclear magnetic resonance analysis, the first spectrum containing individual spectral peaks representative of the one or more metabolite species contained within the biofluid; subjecting each of the individual spectral peaks to a statistical pattern recognition analysis to identify the one or more metabolite species contained within the biofluid; and identifying the one or more metabolite species contained within the biofluid by analyzing the individual spectral peaks of the spectra.

In another embodiment, the present disclosure is directed to a method for detecting breast cancer status within a biofluid. The method comprises measuring one or more metabolite species within the biofluid by subjecting the biofluid to a nuclear magnetic resonance analysis, the analysis producing a spectrum containing individual spectral peaks representative of the one or more metabolite species contained within the biofluid; subjecting the individual spectral peaks to a statistical pattern recognition analysis to identify the one or more metabolite species contained within the biofluid; and correlating the measurement of the one or more metabolite species with a breast cancer status; wherein the one or multiple metabolite species is selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine and combinations comprising at least one of the foregoing.

Yet another embodiment is directed to a method for detecting breast cancer status within a biofluid. The method comprises measuring one or more metabolite species within the sample by subjecting the sample to an analysis that produces a spectrum containing individual spectral peaks representative of the one or more metabolite species contained within the sample; subjecting the individual spectral peaks to a statistical pattern recognition analysis to identify the one or more metabolite species contained within the sample; and correlating the measurement of the one or more metabolite species with a breast cancer status; wherein the one or multiple metabolite species is selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine and combinations comprising at least one of the foregoing.

According to each of the foregoing methods, the statistical pattern recognition analysis can comprise a principal component analysis, a p-value analysis, or a supervised statistical pattern recognition analysis.

Another aspect of the disclosure is a biomarker for detecting breast cancer. The biomarker comprises one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing. In certain embodiments, the biomarker is contained in a biofluid.

Another aspect of the disclosure is the use of the foregoing biomarker for predicting the recurrence of breast cancer in a subject.

Another aspect of the disclosure is the use of the foregoing biomarker for predicting the responsiveness to one or more selected breast cancer therapies in a subject having breast cancer.

Another aspect of the disclosure is a method for predicting the responsiveness to one or more selected breast cancer therapies in a breast cancer subject, comprising measuring the concentration of one or more biomarkers in a biofluid of the subject, wherein the biomarker comprises one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

Another aspect of the disclosure is a method for predicting the absence of any breast cancer in a subject, comprising measuring the concentration Of one or more biomarkers in a biofluid of the subject, wherein the biomarker comprises one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

### BRIEF DESCRIPTION OF THE DRAWINGS

The present teachings will become more apparent and better understood with reference to the following description of exemplary embodiments taken in conjunction with the accompanying drawings, in which corresponding reference characters indicate corresponding parts throughout the several views:

FIGS. 1-4 show a table (Table A) listing metabolite species that were identified as being related to breast cancer using methods according to the present disclosure;

FIG. 5A shows the NMR spectrum of a serum sample from a patient having breast cancer;

FIG. 5B shows the NMR spectrum of a serum sample from a healthy patient;

FIG. 6A shows PCA score plots from a multivariate analysis of NMR measurements from the Cureline samples used in Example 1;

FIG. 6B shows PCA score plots from a multivariate analysis of NMR measurements from the Asterand samples used in Example 1;

FIG. 7 shows a 2D presentation of the PC2 loading of exemplary NMR data from the Cureline samples in accordance with the present teachings;

FIG. 8A shows PLS-DA score plots from a multivariate analysis of NMR measurements from the Cureline samples used in Example 1;

FIG. 8B shows PLS-DA score plots from a multivariate analysis of NMR measurements from the Asterand samples used in Example 1;

FIG. 9 shows a 2D presentation of the LV2 loading of the Cureline samples used in Example 1;

FIG. 10 shows a PLS-DA model and predicted scores from cross validation for the breast cancer and healthy normal samples used in Example 2;

FIG. 11 shows an ROC from the training set of samples shown in FIG. 10;

FIG. 12 shows PLS-DA predicted scores for the testing set of 202 breast cancer and healthy normal serum samples;

FIG. 13 shows an ROC plot derived from the PLS-DA results shown in FIG. 12;

FIG. 14 shows an ROC plot derived from the PLS-DA results using only three biomarkers, formic acid, histidine and 3-hydroxybutyrate; and

FIG. 15 shows an ROC from the PLS-DA analysis of breast cancer and normal patients ages 40 and under.

### DETAILED DESCRIPTION

The present disclosure is related generally to the metabolomics-based analysis of human biological fluids (“biofluid”) to identify metabolite species or sets of metabolite species that function as biomarkers for detecting early forms of breast cancer and for predicting the recurrence of breast cancer. “Biofluid,” as used herein, means any human body fluid and/or fluid extracted from a human body as a fluid, and does not include fluids that are the result of, for example, the digestion of tissues, and the like. Examples of the foregoing include, but are not limited to, bile, blood, blood serum, breath condensate, cerebral spinal fluid, nipple aspirate, plasma, saliva, serum, spinal fluid, tear duct fluid, tissue extracts, urine, and the like.

The biomarkers may be identified by analyzing and comparing biofluid samples from breast cancer patients and matched healthy controls, which may be performed in parallel. Based on the identification and concentration of the biomarkers in a sample of biofluid from a subject, the subject may be classified into a group such as “healthy subject,” “primary breast cancer subject” or “breast cancer recurrence subject.”

In accordance with the present method, a biofluid may be analyzed to produce a spectrum containing individual spectral peaks that are representative of the metabolite species contained within the sample. Suitable techniques for analyzing the biofluid include, but are not limited to, nuclear magnetic resonance (“NMR”), mass spectrometry (following liquid chromatography, gas chromatography, capillary electrophoresis, or atmospheric sample introduction methods such as desorption electrospray ionization, direct analysis in real time, extractive electrospray ionization, etc.), immunoassay enzymatic reactions and Raman spectroscopy. For ease of discussion, NMR will be used throughout the discussion, it being understood that any of the other foregoing methods may be used in place of NMR.

According to the method, biofluids samples are obtained, and NMR measurements are conducted on the biofluids, followed by an advanced statistical pattern recognition analysis (“SPRA”), which can be used to identify the metabolite species contained within the sample. The SPRA also allows sample differentiation by measuring multiple metabolite species in parallel. Multivariate statistical methods, such as principal component analysis (“PCA”), may be applied to reduce the data set size and complexity. Supervised statistical methods that may be used include, but are not limited to, partial least squares discriminant analysis (“PLS-DA”), orthogonal signal correction partial least squares discriminant analysis (“OSC-PLS-DA”), or p-values. Both supervised and unsupervised SPRA, and combinations thereof, may be applied to each of the individual spectral peaks to identify the metabolite species contained within the sample.

After the metabolite species within the biofluid have been subjected to SPRA, individual peaks that show significantly altered concentrations in the spectra from breast cancer patients may be analyzed to identify the metabolite species. Validation of the identified metabolite markers using additional biofluids comprising a test sample set can be preformed, if desired.

Compounds showing significantly altered concentrations in breast cancer samples can be identified and compared to healthy controls using a database of chemical shift values corresponding to known metabolites, which can be confirmed using authentic compounds. H-NMR and statistical analysis of the same samples can then be used to produce additional molecules of interest, as well as to classify subjects, as described, above.

The foregoing exemplary methods have demonstrated that certain perturbations in the glycerophospholipid metabolism, glycolysis, and several amino acid metabolism pathways are related to carcinogenesis.

The foregoing exemplary methods have also been used to identify certain biomarkers (shown in Table A) that have been shown to be related to breast cancer including, but not limited to, acetoacetate, alanine, arginine, asparagine, beta-hydroxybutyrate, creatinine, formate, glucose, glutamine, histidine, isoleucine, methionine, N-acetylaspartate, N-acetylglutamate, proline, threonine, tyrosine, valine, and combinations thereof. Thus, one aspect of the present disclosure is a biomarker comprising one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

The presence or absence, or combination of the presence or absence of the foregoing biomarkers can be used for various predictive purposes. For example, the presence selected biomarkers that are known to be related to certain types of breast cancer, or to a particular occurrence of breast cancer in a particular subject, when present in the biofluid of the subject, can be used to predict the recurrence of breast cancer in the subject. In another example, the absence of certain biomarkers from the biofluid of a subject, that are known to be related to certain types of breast cancer, can be used to predict the absence of any breast cancer in a subject. In another example, the presence of certain biomarkers that are known to be or determined to be responsive to selected breast cancer therapies may be used to predict whether a subject having breast cancer will be responsive to the therapy, should a biofluid sample from the subject contain such biomarkers.

The methods, and advantages and improvements of methods according to the present disclosure are demonstrated in the following examples, which are illustrative only and not intended to limit or preclude other embodiments of the disclosure.

### WORKING EXAMPLES

### Exampple 1

Commercial human blood serum samples from were purchased from two commercial sources. A total of 147 blood serum samples were purchased: 107 samples from Cureline (San Francisco, Calif.) and 40 samples from Asterand (Detroit, Mich.). The 147 serum samples consisted of breast cancer patients (n=74) and gender and age-matched healthy controls (n=73). All the serum samples were obtained from female volunteers of ages ranging from 40 to 75 years old. Samples were stored at −80 ° C. until the measurements were made.

NMR measurements were performed on a Bruker DRX 500 MHz spectrometer equipped with a room temperature HCN probe. Samples were prepared by first vortexing and centrifuging a 530 microliter (“μl”) serum sample that was placed into a standard 5 millimeter (“mm”) NMR tube for analysis. A 100 μl solution of 1.5 millimolar (“mM”) 3-(trimethylsilyl) propionic-(2,2,3,3-d₄) acid sodium salt (“TSP”) in D₂O injected into a capillary that was placed into the NMR sample tube in a concentric fashion to provide a deuterium frequency lock, with the TSP providing a frequency standard (δ=0.00). Samples were measured using a standard 1D CPMG (Carr-Purcell-Meiboom-Gill) pulse sequence coupled with water presaturation. For each spectrum, 64 transients were collected resulting in 32k data points using a spectral width of 6000 Hertz (“Hz”). . An exponential weighting function corresponding to 0.3 Hz line broadening was applied to the free induction decay (FID) before Fourier transformation. After phasing and baseline correction using Bruker's XWINNMR software, the processed data were saved in ASCII format for further multivariate analysis. The spectral region from 4.5 parts-per-million (“ppm”) to 6 ppm, which contains water and urea signals, was removed from each spectrum prior to data analysis. Spectral alignment was performed using either the TSP signal at 0 ppm or the two alanine peaks near 1.44 ppm.

All pre-processing and multivariate analyses of the experimental data were carried out using Matlab 7.1.0.246 (Mathworks Inc., Natick, MA) with the PLS toolbox (Eigenvector Research Inc, Wenatchee, Wash.). NMR data were transferred from the instruments in plain text format and then imported into Matlab.

NMR spectra were used at full resolution (16K frequency buckets of equal width). The NMR data were normalized against the total spectral intensity and then mean-centering was carried out prior to multivariate analysis.

PCA was performed using the PLS toolbox to identify metabolite signals. The NMR data was also analyzed using p-values after total spectral intensity normalization or by using the integrated TSP signal for normalization to identify additional putative biomarkers.

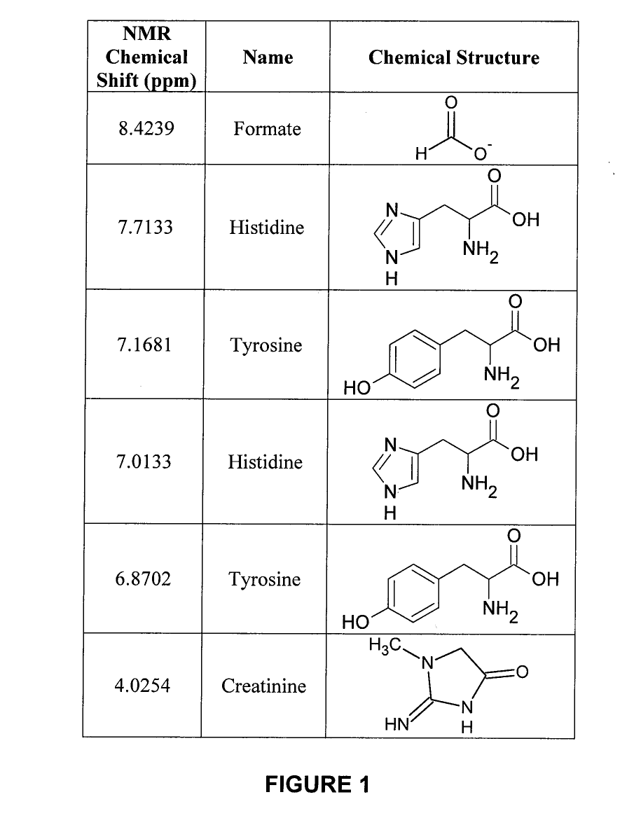

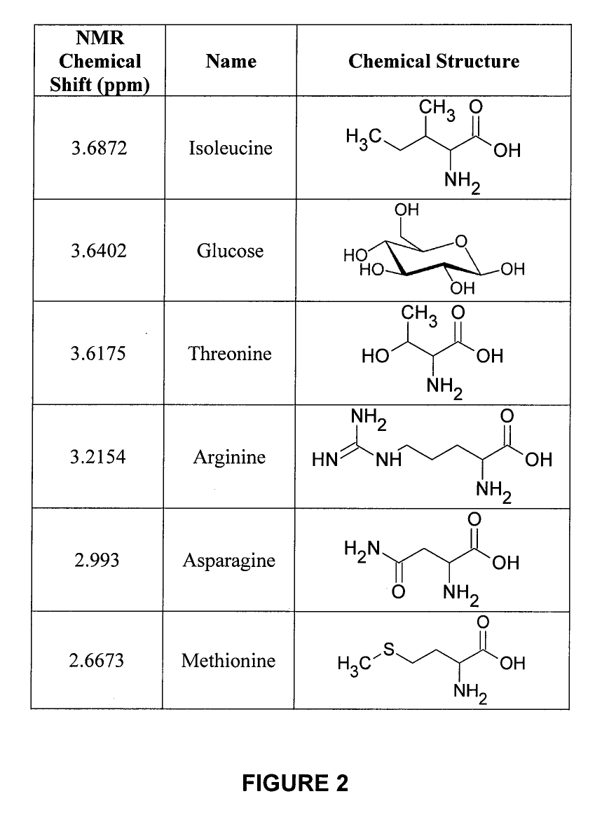

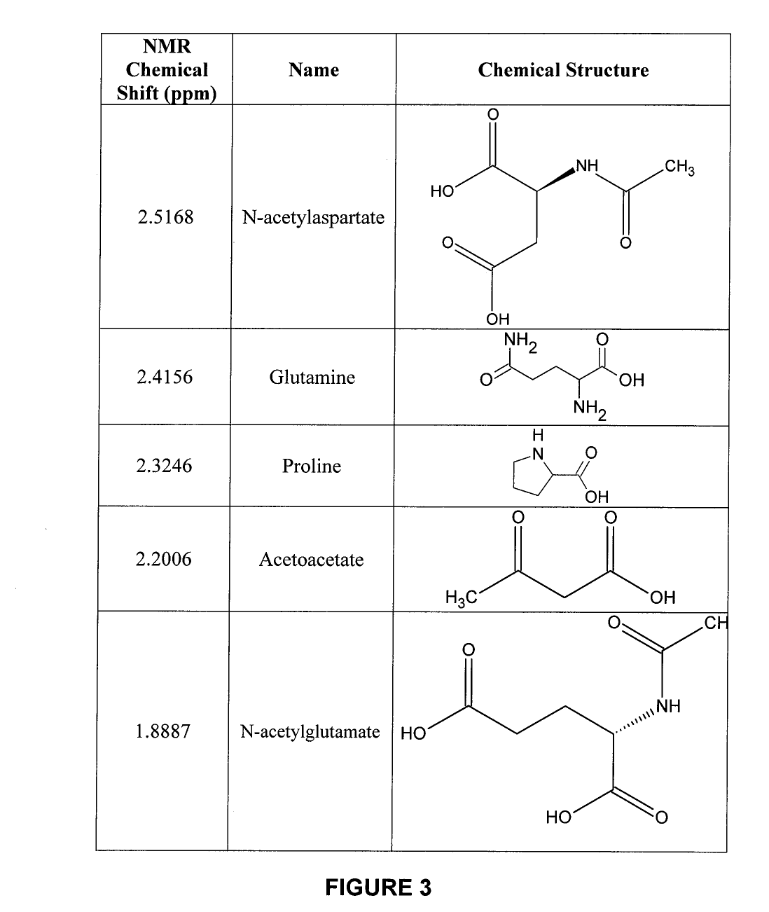

PLS-DA was then used to combine multiple metabolites into a statistical model, using the metabolite signals as inputs. Individual sample sets were used to build the model (the “training set”). The entire sample set was split into two halves: a training set for model building; and a “test set” of samples to evaluate the results of the model in terms of sensitivity (percent breast cancer detected correctly) and specificity (percent healthy samples detected correctly). The results of the foregoing are illustrated in FIGS. 1-4 (Table A) and FIGS. 5-10.

FIGS. 5A and 5B are 1D CPMG spectra of breast cancer and normal samples. A visual inspection and comparison of the spectra in FIGS. 5A and 5B shows that the intensities of the peaks in the aromatic region were very small. In addition, a number of peaks appeared to be clearly different between the cancer and normal samples. For example, the lipid signal near 0.8 ppm was increased in the cancer sample, and the acetoacetate signal was also significantly larger.

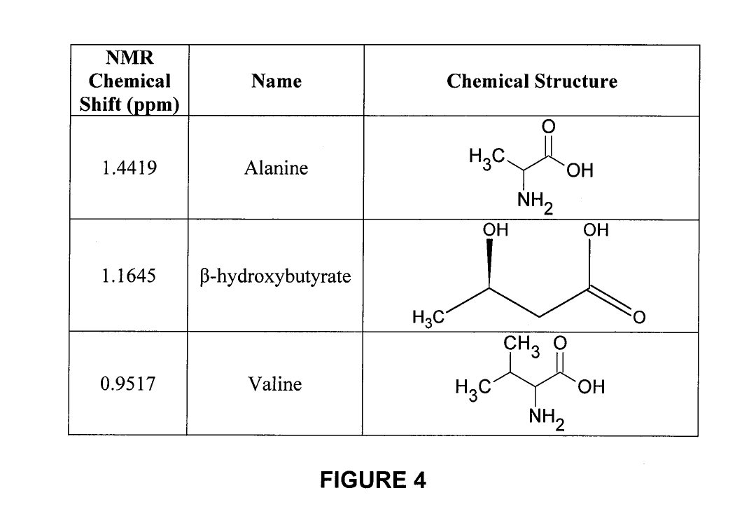

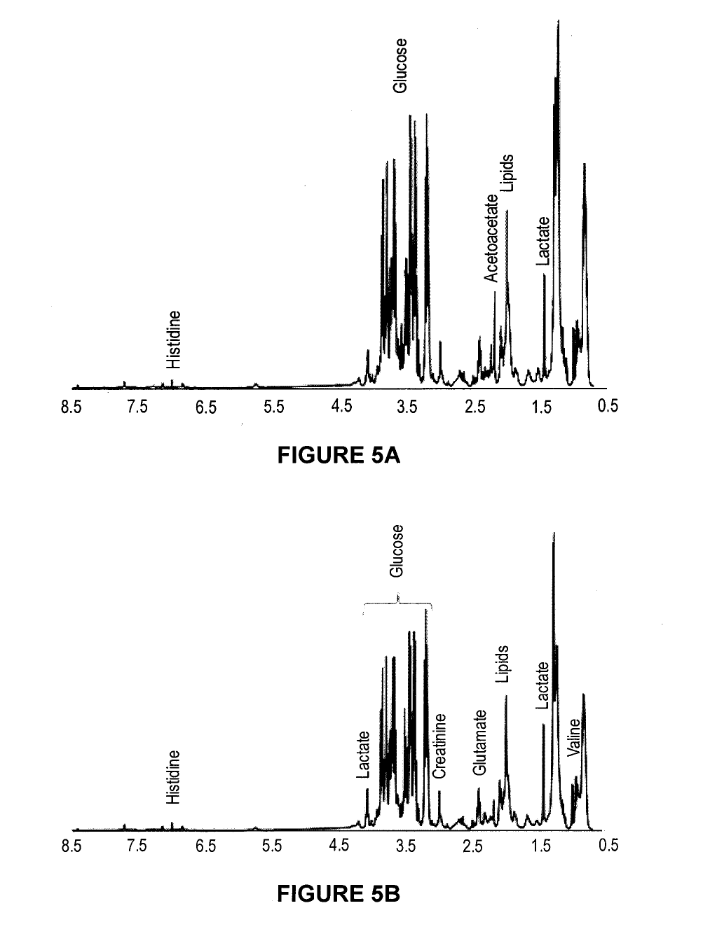

PCA was applied to the NMR spectra from the 107 breast cancer and healthy control samples from Cureline, and the score plot is shown in FIG. 6A.

Similarly, PCA was applied to NMR spectra from the 40 breast cancer and healthy control samples from Asterand, and the score plot is shown in FIG. 6B. All of the samples were classified by projecting corresponding NMR spectra onto the 2D plane of PC1 and PC2. Using a linear discriminate analysis, a discriminant accuracy of approximately 80 to 90% was achieved. The separation occurred along the first two PC's, primarily along PC2 in the first sample set (from Cureline), and along PC1 for the second, smaller sample set (from Asterand).

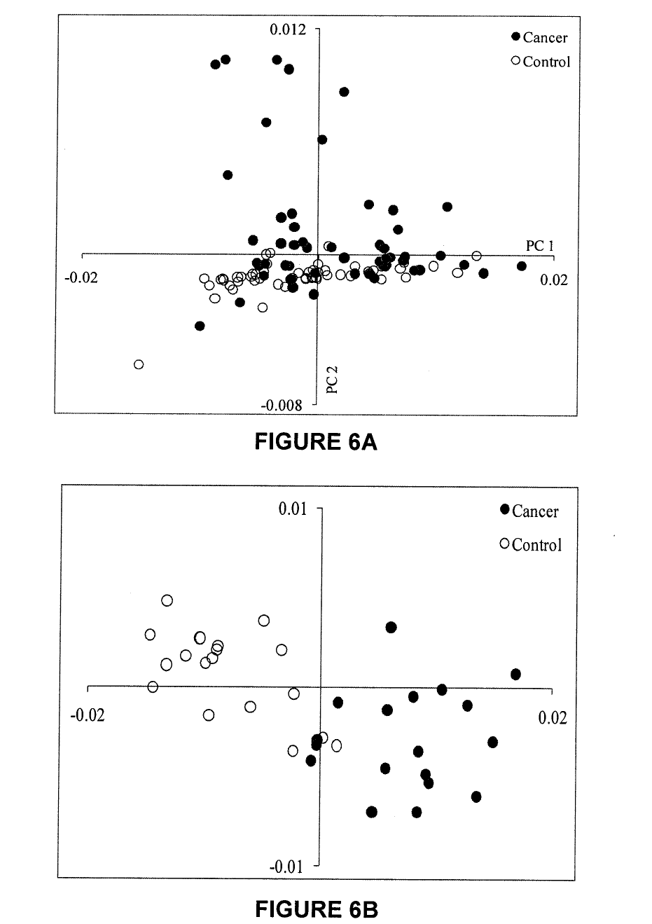

Several putative biomarkers responsible for the separation were identified from the PCA loading plot for PC2 shown in FIG. 7. As shown, the levels of glucose (3.53 ppm, 3.73 ppm and 3.86 ppm) were observed to decrease in the serum samples from the breast cancer patients. In addition, lipid signals at 0.92 ppm and 1.31 ppm were more concentrated in the serum samples from the breast cancer patients. The PC1 loading was mainly contributed by the variation within the breast cancer patients, for instance, the concentration of sugars and lipids.

Metabolites identified from the PCA loading plots and p-values analysis are shown in Table A (FIGS. 1-4).

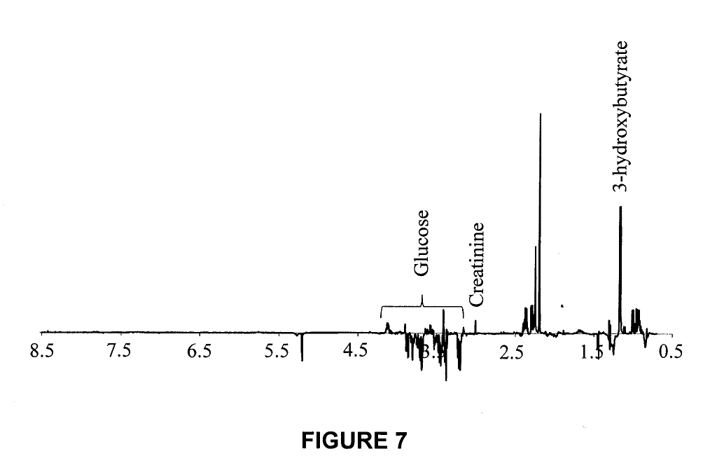

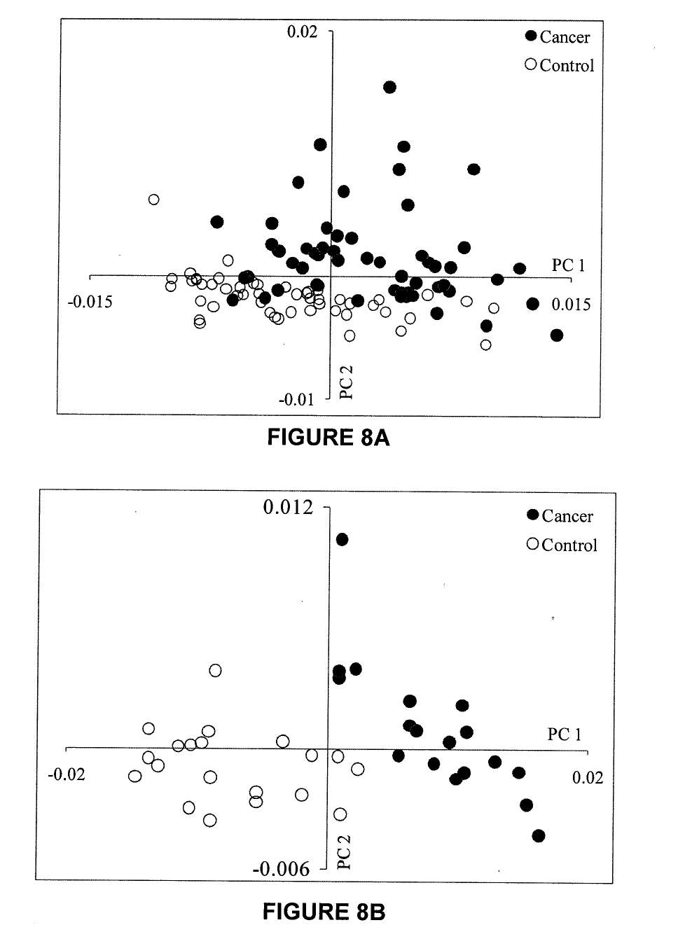

FIGS. 8A and 8B are score plots resulting from the supervised PLS-DA analysis of the same samples. The score plots illustrate the improved separation that was achieved using this approach. Again, the first sample set (Cureline) was better separated, while the second sample set (Asterand) was completely separated along the x-axis.

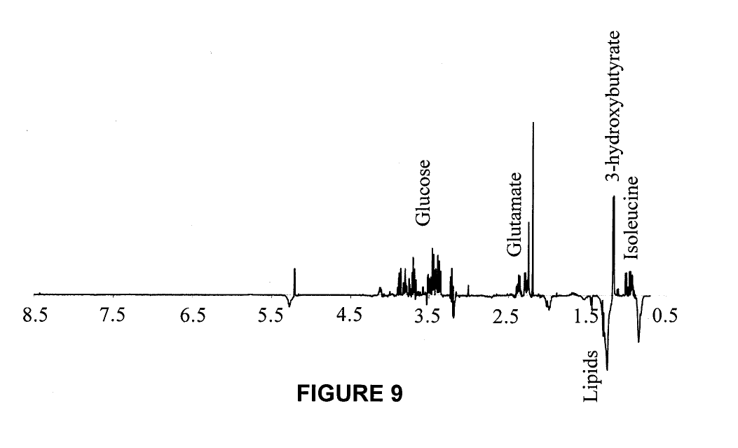

The loading plot shown in FIG. 9 is that for the second latent variable, LV2 which corresponds to the y-axis in FIG. 8A. Several additional metabolites were identified using this approach, including glutamine, isoleucine and 3-hydroxybutyrate.

### Example 2

Commercial human blood serum samples from breast cancer patients (n=142) and gender and age-matched healthy controls (n=264) were purchased from four commercial sources (Cureline, Asterand, Innovative Research and SeraCare). All of the serum samples were obtained from female volunteers of ages ranging from 18 to 79 years old. Samples were stored at −80 ° C. until the measurements were made.

The NMR measurements were measured in the same manner as in Example 1, as was the multivariate analysis of the NMR spectra.

NMR spectral peaks corresponding to the biomarkers listed above and shown in Table A were individually integrated and the data were then normalized against the total spectral intensity.

The samples were split into two sets consisting of a training set containing 72 cancer patient samples and 132 healthy patient samples from the four commercial sources and a testing set consisting of 70 cancer and 132 healthy patients from the same sources.

Using the integrated and normalized spectral peaks of the 18 metabolites biomarkers discovered in Example 1 and as described in Table A, PLS-DA was used to combine multiple metabolites into a statistical model based on the training set of samples, using the metabolite signals as inputs. Leave one out cross validation was used to evaluate the model and 3 latent variables (“LVs”) were selected according to the root-mean-square error of cross validation (“RMSECV”) curve. The results of the foregoing are illustrated in FIGS. 6-10.

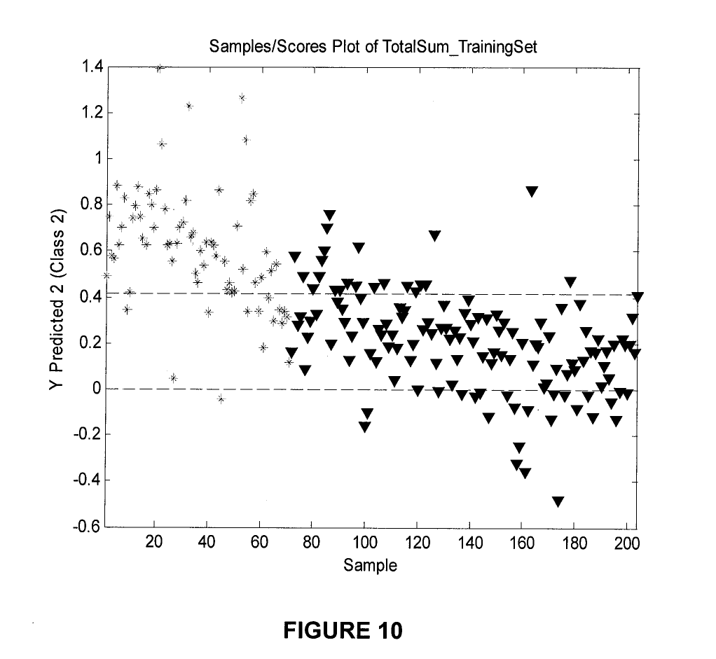

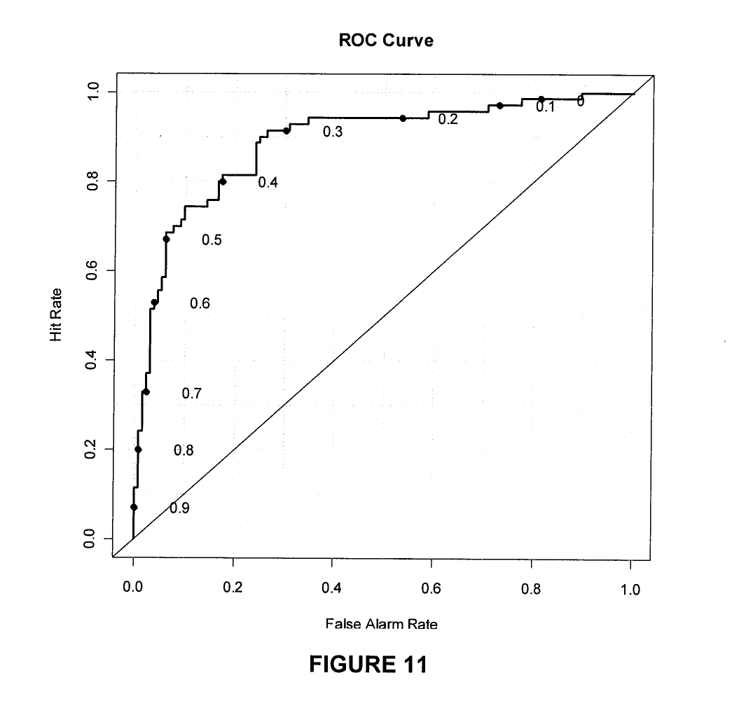

The cross validation results are shown FIG. 10, with the cancer patient samples indicated by the asterisks and the healthy patient samples indicated by the triangles. The predictive ability of the model based on a cut off value of 0.5 was determined to be 0.900 from the area under the curve. The sensitivity specificity for detecting breast cancer by NMR and based on this model is approximately 85% and 84%, respectively. Alternatively, at a specificity of 94%, this approach yields a sensitivity of 70%. The results of the training set model are shown in the ROC plot of FIG. 11.

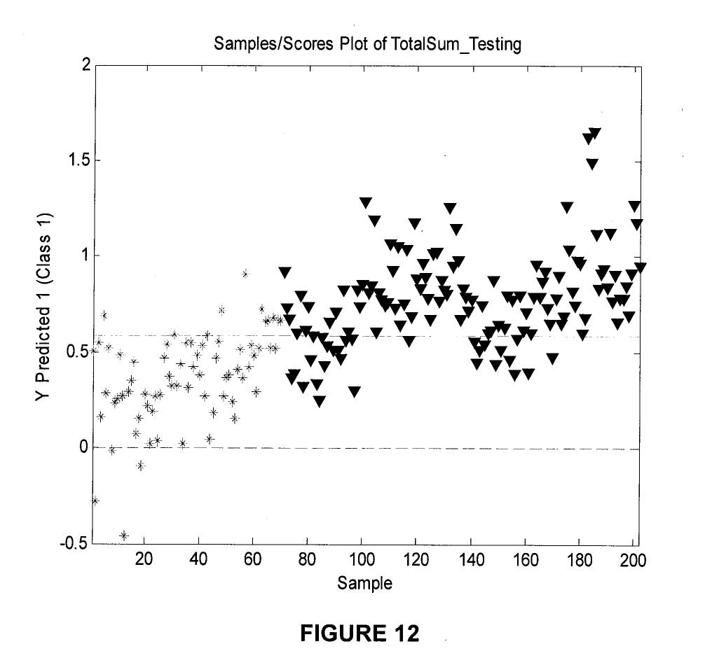

After training the statistical model, the model was used to predict the remaining samples using the testing set. The results of the model are shown in FIG. 12, again with the cancer patient samples indicated by the asterisks and the healthy patient samples indicated by the triangles.

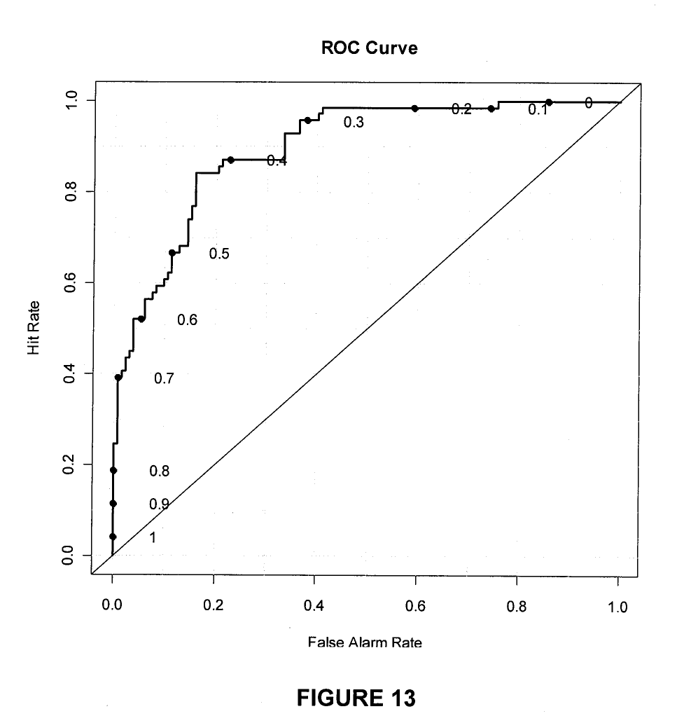

FIG. 13 shows the ROC plot indicating the overall sensitivity (percent breast cancer detected correctly) and specificity (percent healthy samples detected correctly) achieved using the model. The sensitivity and specificity for detecting breast cancer by NMR and based on this model was approximately 85% and 84%, respectively.

It can also be seen in FIG. 13 that approximately 58% of the samples were correctly identified as showing the absence of breast cancer in a subject, i.e., breast cancer-free subjects, while only missing 2% of the patient samples that do in fact have cancer. This approach may be of utility to rule out cancer cases in a majority of women. The terms “absence of breast cancer” and “breast cancer-free” are used interchangeably herein, and mean that the presence of breast cancer is not detectable.

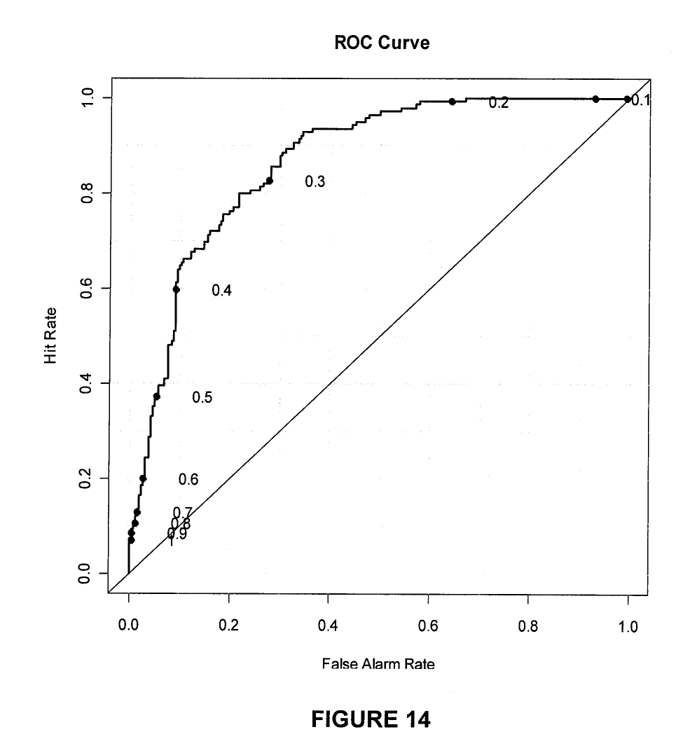

Reducing the model to the three biomarkers, namely formic acid, histidine and 3-hydroxibutyrate, the PLS-DA model developed as described above yields a specificity of 50% and a sensitivity of 97%, which can be seen in the ROC plot of FIG. 14. In other words, 50% of women can be said to have no breast cancer, while missing only 3% of patients that do in fact have cancer. This corresponds to approximately 0.06% of women who would be misdiagnosed using this approach.

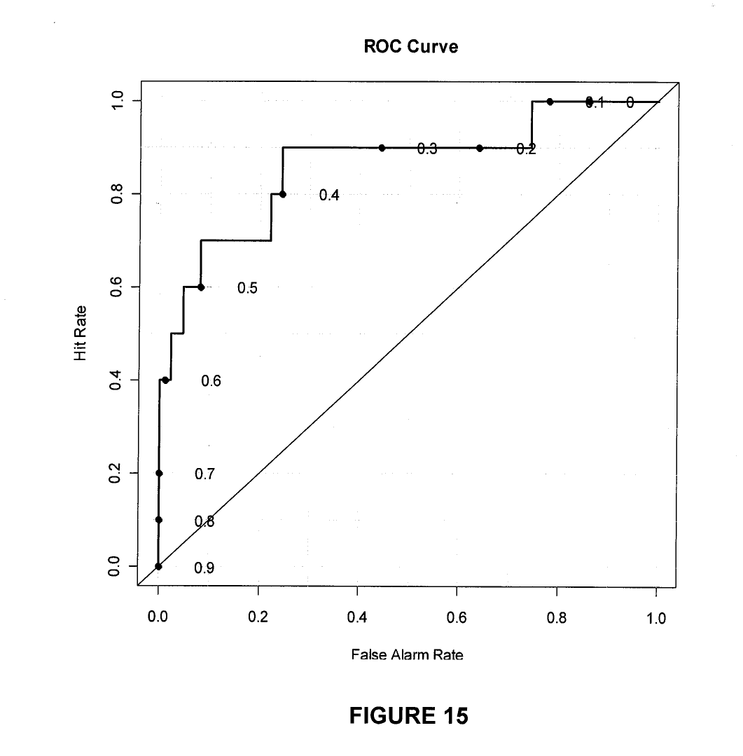

Finally, the ROC plot results from applying the metabolite profiling model for breast cancer patients and healthy controls in subjects 40 years old or younger is shown in FIG. 15. The ROC plot indicates that the metabolite profile determined using NMR detected biomarkers provided excellent sensitivity (about 70%) at a specificity level of approximately 92%.

In addition to the exemplary compounds listed above, it should be understood and appreciated herein that other metabolite species useful as biomarkers may also be identified in accordance with the present disclosure. Some of these additional metabolite species include, but are not limited to lipid signals near 0.8 and 1.2 ppm, lactic acid, urea, glutamate, lysine, creatine and isobutyrate.

Throughout the detailed description, it should be noted that the terms “first,” “second,” and the like herein do not denote any order or importance, but rather are used to distinguish one element from another, and the terms “a” and “an” herein do not denote a limitation of quantity, but rather denote the presence of at least one of the referenced items. Similarly, it is noted that the terms “bottom” and “top” are used herein, unless otherwise noted, merely for convenience of description, and are not limited to any one position or spatial orientation. In addition, the modifier “about” used in connection with a quantity is inclusive of the stated value and has the meaning dictated by the context (e.g., includes the degree of error associated with measurement of the particular quantity). Compounds are described using standard nomenclature. For example, any position not substituted by an indicated group is understood to have its valency filled by a bond as indicated, or a hydrogen atom A dash (“-”) that is not between two letters or symbols is used to indicate a point of attachment for a substituent. Unless defined otherwise herein, all percentages herein mean weight percent (“wt. %”). Furthermore, all ranges disclosed herein are inclusive and combinable (e.g., ranges of “up to about 25 wt. %, with about 5 wt. % to about 20 wt. % desired, and about 10 wt. % to about 15 wt. % more desired,” are inclusive of the endpoints and all intermediate values of the ranges, e.g., “about 5 wt. % to about 25 wt. %, about 5 wt. % to about 15 wt. %,” etc.). The notation “+/−10%” means that the indicated measurement may be from an amount that is minus 10% to an amount that is plus 10% of the stated value. Finally, unless defined otherwise herein, technical and scientific terms used herein have the same meaning as is commonly understood by one of skill in the art to which this disclosure belongs.

The embodiments of the present disclosure described above are not intended to be exhaustive or to limit the disclosure to the precise forms disclosed in the detailed description. Rather, the embodiments are chosen and described so that others skilled in the art may appreciate and understand the principles and practices of the present disclosure.

While an exemplary embodiment incorporating the principles of the present disclosure has been disclosed hereinabove, the present disclosure is not limited to the disclosed embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A method for the parallel identification of one or more metabolite species within a biofluid, comprising:
producing a first spectrum by subjecting the biofluid to a nuclear magnetic resonance analysis, the first spectrum containing individual spectral peaks representative of the one or more metabolite species contained within the biofluid;
subjecting each of the individual spectral peaks to a statistical pattern recognition analysis to identify the one or more metabolite species contained within the biofluid; and
identifying the one or more metabolite species contained within the biofluid by analyzing the individual spectral peaks of the spectra.

2. The method of claim 1, wherein subjecting each of the individual spectral peaks to the statistical pattern recognition analysis comprises subjecting the spectral peaks to a principal component analysis.

3. The method of claim 1, wherein subjecting each of the individual spectral peaks to the statistical pattern recognition analysis comprises subjecting the spectral peaks to a p-value analysis.

4. The method of claim 1, wherein subjecting each of the individual spectral peaks to the statistical pattern recognition analysis comprises subjecting the individual spectral peaks to a supervised statistical pattern recognition analysis.

5. The method of claim 1, further comprising assigning the biofluid into a defined class after identifying the one or more metabolite species contained in the biofluid.

6. The method of claim 1, further comprising determining the concentration of the one or more metabolite species contained in the biofluid.

7. The method of claim 1, wherein the one or more metabolite species are adapted to function as breast cancer biomarkers.

8. The method of claim 1, wherein the one or more metabolite species are selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

9. A method for detecting breast cancer status within a biofluid, comprising:
measuring one or more metabolite species within the biofluid by subjecting the biofluid to a nuclear magnetic resonance analysis, the analysis producing a spectrum containing individual spectral peaks representative of the one or more metabolite species contained within the biofluid;
subjecting the individual spectral peaks to a statistical pattern recognition analysis to identify the one or more metabolite species contained within the biofluid; and
correlating the measurement of the one or more metabolite species with a breast cancer status;
wherein the one or multiple metabolite species is selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine and combinations comprising at least one of the foregoing.

10. The method of claim 9, wherein subjecting each of the individual spectral peaks to the statistical pattern recognition analysis comprises subjecting the spectral peaks to a principal component analysis.

11. The method of claim 9, wherein subjecting each of the individual spectral peaks to the statistical pattern recognition analysis comprises subjecting the spectral peaks to a p-value analysis.

12. The method of claim 9, wherein subjecting each of the individual spectral peaks to the statistical pattern recognition analysis comprises subjecting the individual spectral peaks to a supervised statistical pattern recognition analysis.

13. The method of claim 9, further comprising assigning the biofluid into a defined class after identifying the one or more metabolite species contained in the biofluid.

14. The method of claim 9, further comprising determining the concentration of the one or more metabolite species contained in the biofluid.

15. The method of claim 9, further comprising determining the concentration of the one or more metabolite species contained in the biofluid.

16. The method of claim 9, wherein the one or more metabolite species are adapted to function as breast cancer biomarkers.

17. A method for detecting breast cancer status within a biofluid, comprising:
measuring one or more metabolite species within the sample by subjecting the sample to an analysis that produces a spectrum containing individual spectral peaks representative of the one or more metabolite species contained within the sample;
subjecting the individual spectral peaks to a statistical pattern recognition analysis to identify the one or more metabolite species contained within the sample; and
correlating the measurement of the one or more metabolite species with a breast cancer status;
wherein the one or multiple metabolite species is selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine and combinations comprising at least one of the foregoing.

18. The method of claim 17, wherein subjecting each of the individual spectral peaks to the statistical pattern recognition analysis comprises subjecting the spectral peaks to a principal component analysis.

19. The method of claim 17; wherein subjecting each of the individual spectral peaks to the statistical pattern recognition analysis comprises subjecting the spectral peaks to a p-value analysis.

20. The method of claim 17, wherein subjecting each of the individual spectral peaks to the statistical pattern recognition analysis comprises subjecting the individual spectral peaks to a supervised statistical pattern recognition analysis.

21. The method of claim 17, further comprising assigning the biofluid into a defined class after identifying the one or more metabolite species contained in the biofluid.

22. The method of claim 17, further comprising determining the concentration of the one or more metabolite species contained in the biofluid.

23. The method of claim 17, further comprising determining the concentration of the one or more metabolite species contained in the biofluid.

24. The method claim 17, wherein the one or more metabolite species are adapted to function as breast cancer biomarkers.

25. The method of claim 17, wherein the analysis method is selected from the group consisting of NMR, mass spectrometry, immunoassay, enzymatic reaction, magnetic resonance, magnetic resonance imaging, Raman spectroscopy, infrared spectroscopy and combinations thereof.

26. A biomarker for detecting breast cancer, comprising one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

27. The biomarker of claim 26, wherein the biomarker is contained in a biofluid.

28. Use of a biomarker according to claim 26, for predicting the recurrence of breast cancer in a subject.

29. Use of a biomarker according to claim 26, for predicting the responsiveness to one or more selected breast cancer therapies in a subject having breast cancer.

30. A method for predicting the responsiveness to one or more selected breast cancer therapies in a breast cancer subject, comprising measuring the concentration of one or more biomarkers in a biofluid of the subject, wherein the biomarker comprises one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.

31. A method for predicting the absence of any breast cancer in a subject, comprising measuring the concentration of one or more biomarkers in a biofluid of the subject, wherein the biomarker comprises one or more metabolite species selected from the group consisting of formate, histidine, tyrosine, creatinine, isoleucine, glucose, threonine, arginine, asparagine, glutamine, methionine, N-acetylaspartate, proline, N-acetylglutamate, alanine, beta-hydroxybutyrate, valine, parts thereof, and combinations comprising at least one of the foregoing.
